(19) United States
(12) Patent Application Publication
(10) Pub. No.: US 2014/0005355 A1
(43) Pub. Date: Jan. 2, 2014

(54) PROCESS FOR PREPARATION OF CASPOFUNGIN ACETATE AND INTERMEDIATES

(71) Applicants: Kiran Kumar Kothakonda, Secunderabad (IN); Sandeep Rao Sripathi, Electronic City (IN); Srinivas Pullela Venkata, Hyderabad (IN); Lijo George, Irinjalakuda (IN); Anegondi Sreenivasa Prasad, Hyderabad (IN)

(72) Inventors: Kiran Kumar Kothakonda, Secunderabad (IN); Sandeep Rao Sripathi, Electronic City (IN); Srinivas Pullela Venkata, Hyderabad (IN); Lijo George, Irinjalakuda (IN); Anegondi Sreenivasa Prasad, Hyderabad (IN)

(73) Assignee: BIOCON LIMITED, Bangalore (IN)

(21) Appl. No.: 13/979,529

(22) PCT Filed: Feb. 18, 2011
(86) PCT No.: PCT/IB2011/050681

§ 371 Date: Jul. 12, 2013

(30) Foreign Application Priority Data
Jan. 3, 2011 (IN) 10/CHE/2011

(51) Int. Cl. C07K 7/54

(52) U.S. Cl. CPC C07K 7/54
USPC 530/323

(57) ABSTRACT

The present invention discloses a process for the preparation of Caspofungin and its intermediates from Pneumocandin B₀.

### TECHNICAL FIELD

The disclosure is directed to processes for preparing Caspofungin and intermediates and their use in preparation of Caspofungin salts thereof.

### BACKGROUND

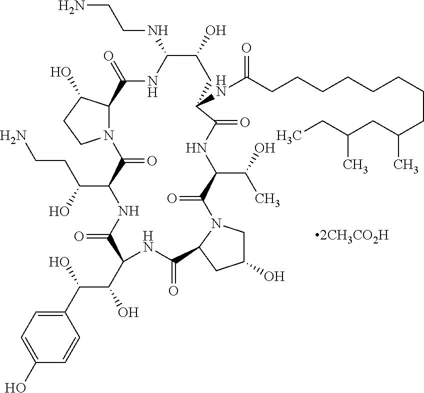 [SMILES: CCC(C)CC(C)CCCCCCCCC(=O)N[C@H]1C[C@@H](O)[C@@H](NCCN)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Caspofungin acetate is 1-[(4R,5S)-5-[(2-aminoethyl)amino]-N2-(10,12-dimethyl-1oxotetradecyl)-4-hydroxy-L-ornithine]-5-[(3R)-3-hydroxy-L-ornithine]pneumocandin B₀ diacetate salt. Caspofungin is sold under the brand name Candid. Caspofungin acetate is indicated in adults and pediatric patients (3 months and older) for empirical therapy for presumed fungal infections in febrile, neutropenic patients; treatment of candidemia and the following Candida infections (intra-abdominal abscesses, peritonitis and pleural space infections); treatment of esophageal candidiasis; and treatment of invasive aspergillosis in patients who are refractory to or intolerant of other therapies.

A number of relevant processes for preparation of Caspofungin and salts thereof are disclosed. Caspofungin, intermediates and their preparation are disclosed in EP 0 630 232. These methods for the preparation of Caspofungin are difficult to adopt during bulk production, more impurities are generated and very tedious to purify. EP 0 912 603 describes the process for the preparation of caspofungin using phenyl boronic acid and thiophenol. The intermediates generated during this reaction are not stable and also resulted in low yields. This patent further teaches the selective deprotection of phenyl boronic acid protection. This process results in tedious byproducts and low yields. Nevertheless, there remains a need in the art for processes of preparing Caspofungin that are both cost effective, have fewer purification steps, and/or result in higher purity of the final product, thereby making them more suitable for industrial scale preparation. Meanwhile, during the process of investigation for the robust process for bulk synthesis the new process was identified. The new process for the preparation of Caspofungin is minimizing the formation of the impurities and relatively less tedious and results in better yields.

### STATEMENT OF DISCLOSURE

Accordingly, the present disclosure is in relation to a process for preparation of Caspofungin, said process comprising acts of:

(i). conversion of pneumocandin B₀ to compound of formula II in a suitable solvent,

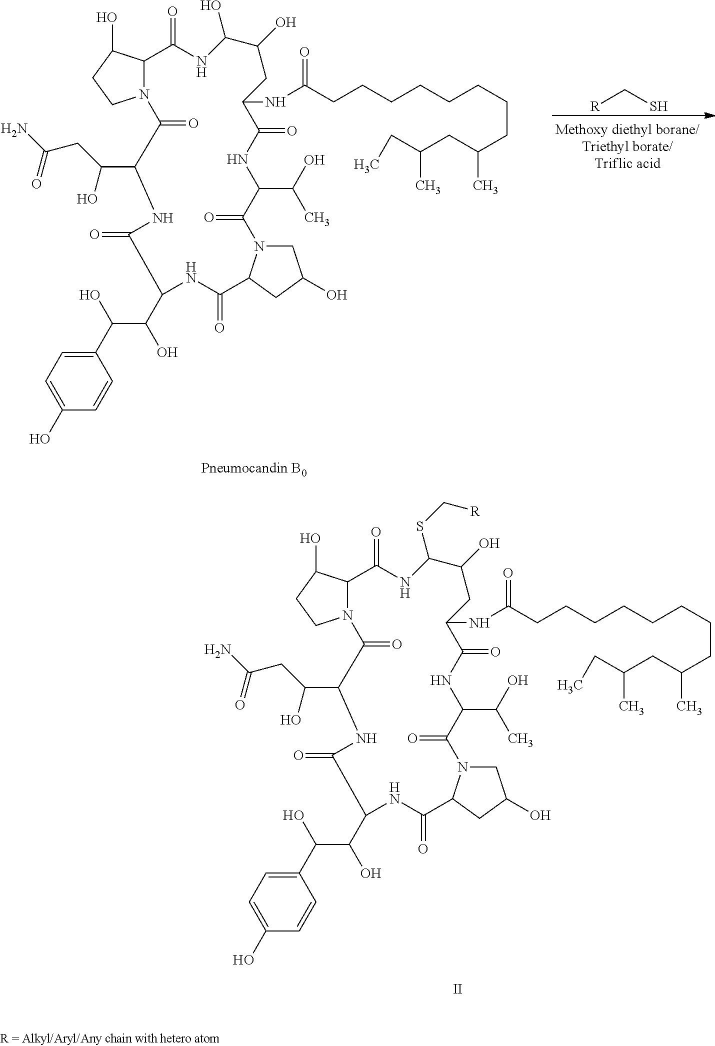

(ii). conversion of formula II to III in a suitable solvent,

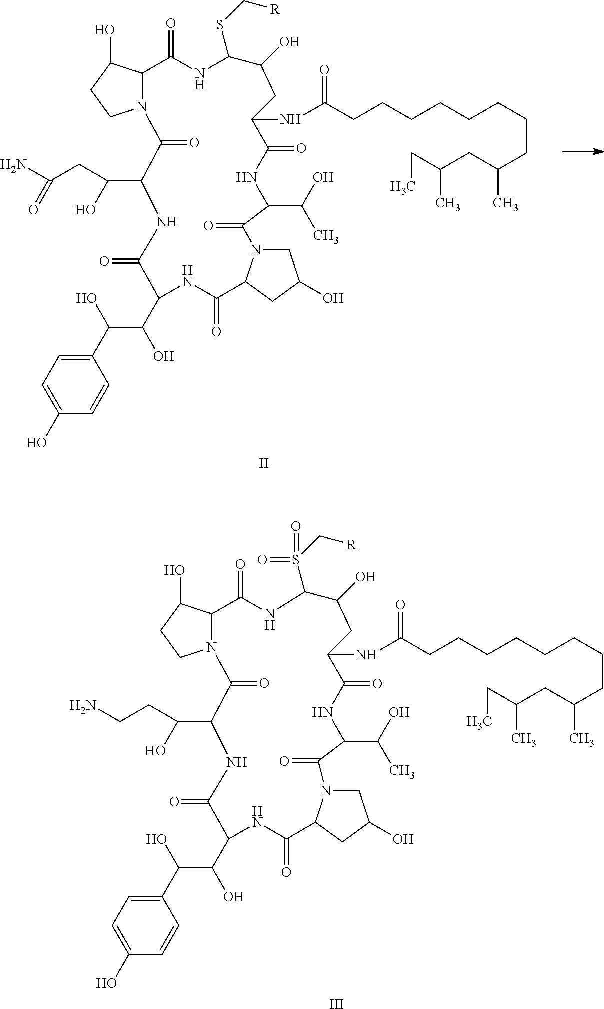

(iii). conversion of formula III to formula IV in a suitable solvent,

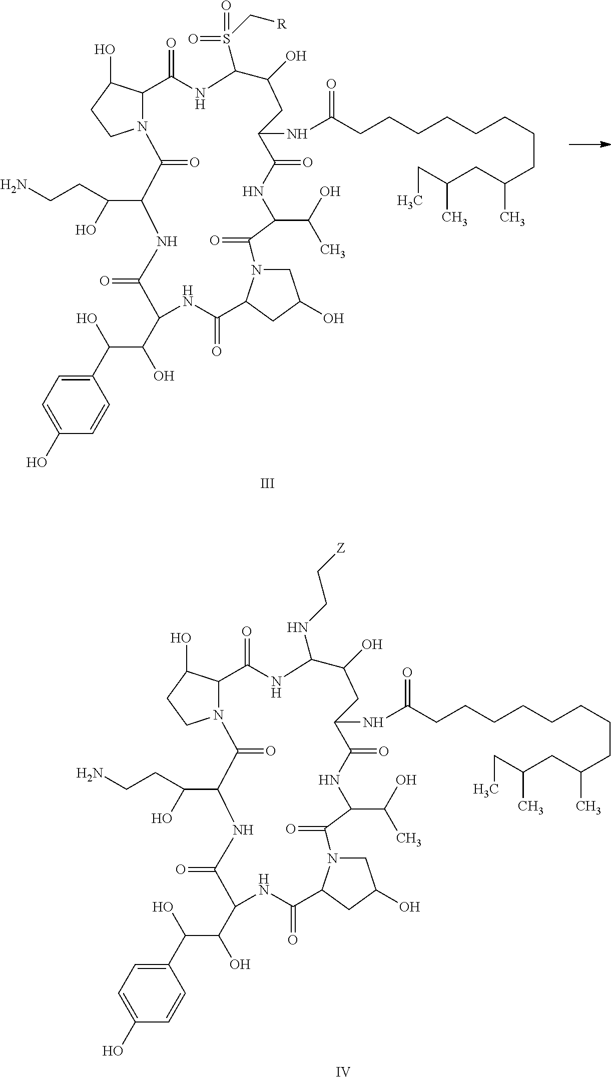

Where Z is NHBOC or N3
   (iv). Optionally reacting compound IV with BOC deprotecting reagent in a suitable solvent to isolate Caspofungin salt wherein Z is NHBOC
   (v). Optionally hydrogenating compound of formula IV in a suitable solvent to isolating the caspofungin salt wherein Z is N₃;
   a compound of formula II,

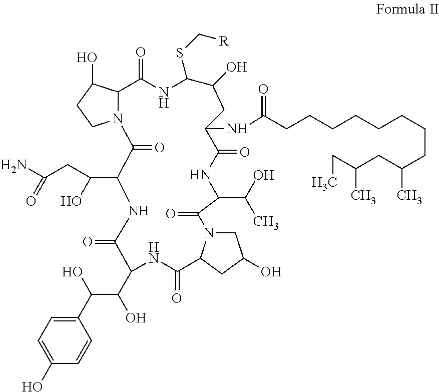

Wherein, R=any side chain consisting of heteroatom;

a compound of formula III,

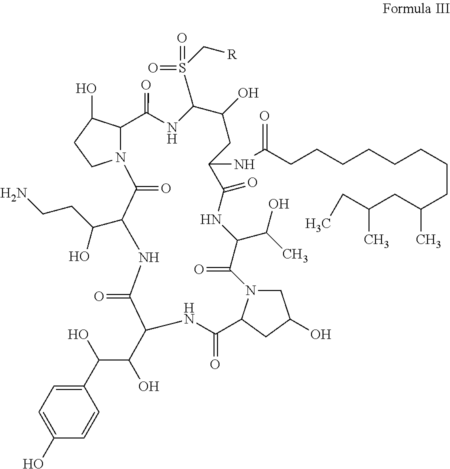

Wherein, R is any side chain consisting of heteroatom.

a compound of formula IV,

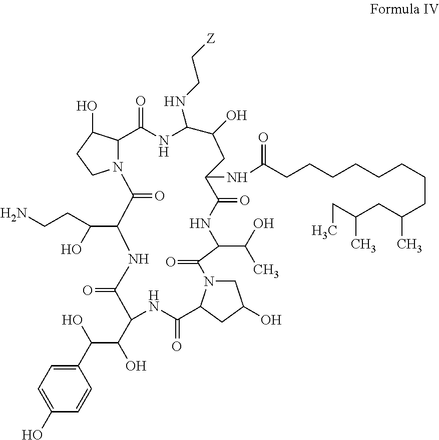

Wherein, Z═NHBOC or N₃.

### BRIEF DESCRIPTION OF THE FIGURES

FIG. 1: Provides a process where pneunocandin B₀ or derivatives are reacted with thiol compounds in anhydrous condition. This reaction is carried out by using the reagents methoxy diethyl borane or triethyl borate or mixtures thereof and triflic acid. The resulted compound is optionally reduced by employing boron dimethyl sulphide complex and further oxidized to get the sulphone of compound III. The reaction further proceeded by reacting the resulted intermediate with N-Boc ethylene diamine and followed by deprotected to form Caspofungin.

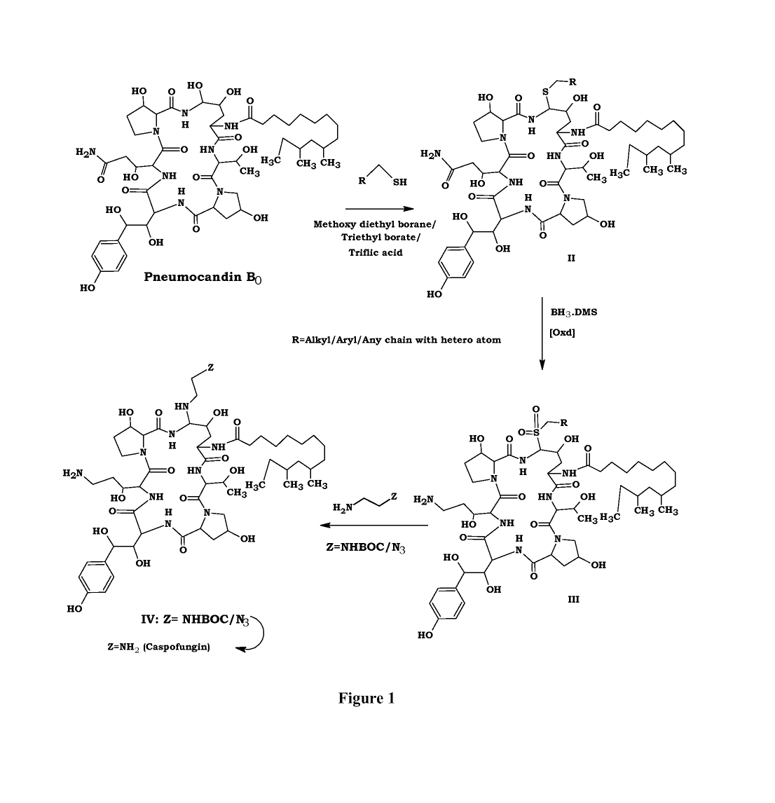

FIG. 2: Provides a procees where pneumocandin B₀ is reduced using borane dimethylsulfide complex and treated with metal halide in the presence of a lewis acid to result compound of formula V which is further treated with N-Boc ethylene diamine followed by deprotection yields pure Caspofungin.

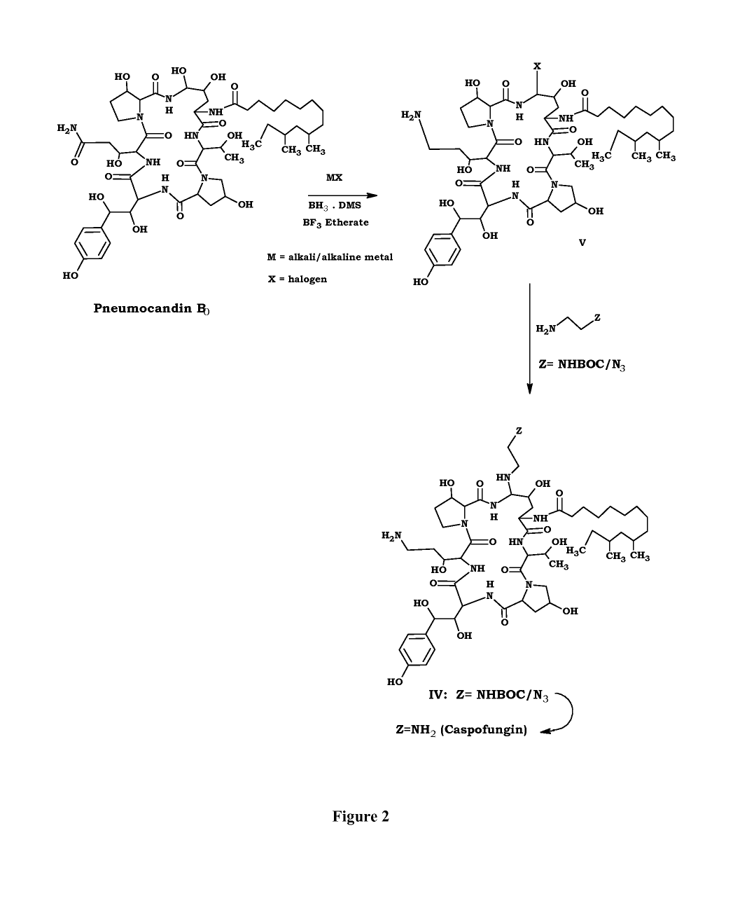

### DETAILED DESCRIPTION OF THE DISCLOSURE

The present disclosure relates to a process of preparation of Caspofungin, said process comprising acts of:

(i). conversion of pneumocandin B₀ to compound of formula II in a suitable solvent,

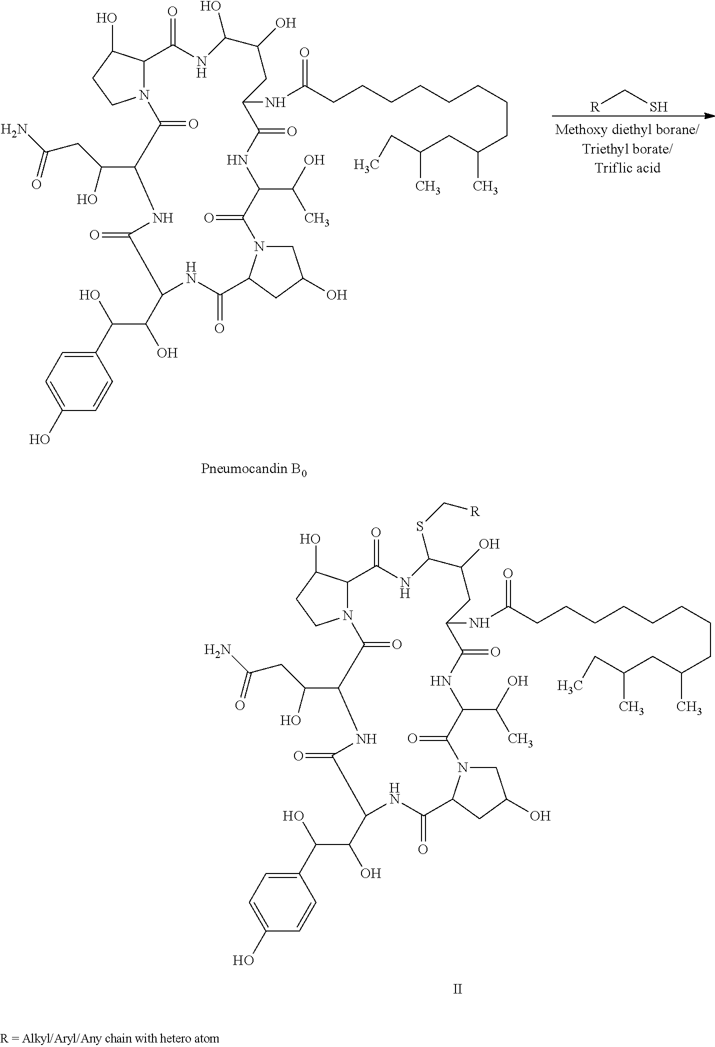

(ii) conversion of formula II to III in a suitable solvent,

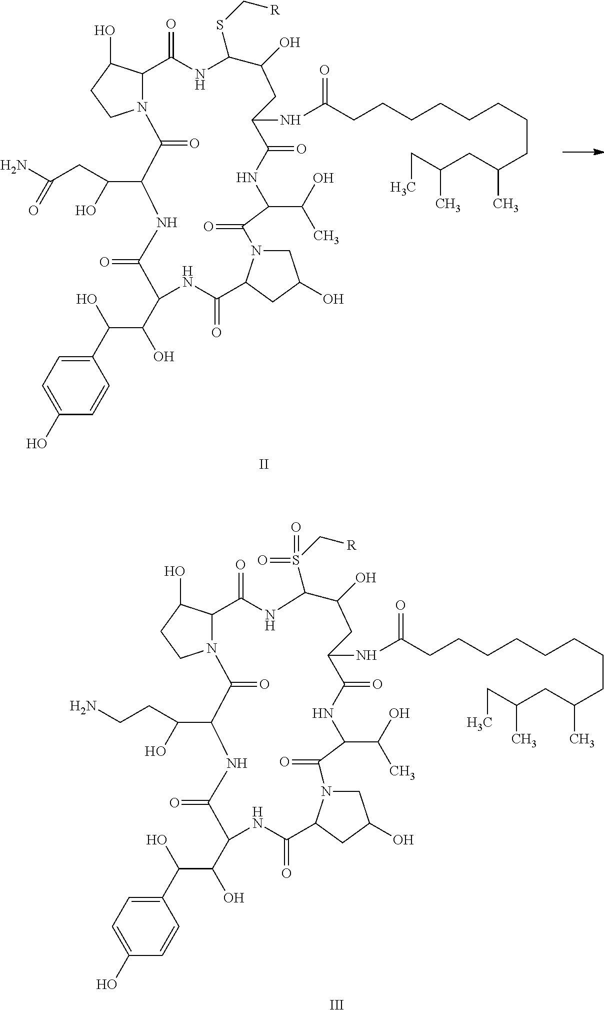

(iii) conversion of formula III to formula IV in a suitable solvent,

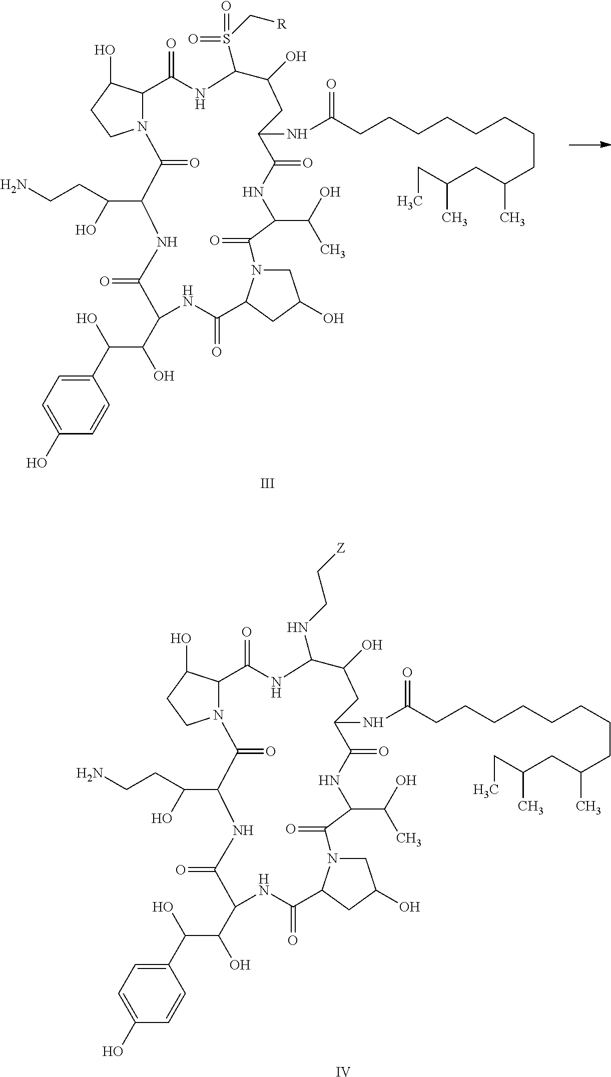

Where Z is NHBOC or N3
   (iv) Optionally reacting compound IV with BOC deprotecting reagent in a suitable solvent to isolate Caspofungin salt wherein Z is NHBOC
   (v). Optionally hydrogenating compound of formula IV in a suitable solvent to isolating the caspofungin salt wherein Z is N₃.

In an embodiment of the present disclosure, pneumocandin B₀ is reacted with RCH2SH in presence of alkoxy dialkylborane or trialkyl borate or mixture thereof, and triflic acid in a suitable solvent to obtain compound of the formula II.

In another embodiment of the present disclosure, R is selected from a group comprising alkyl, aryl or any chain comprising a hetero atom, or mixture thereof.

In yet another embodiment of the present disclosure, alkoxy dialkylborane is methoxy diethylborane.

In still another embodiment of the present disclosure, trialkyl borate is triethyl borate.

In still another embodiment of the present disclosure, conversion of compound II to compound III further comprises.

a) reducing compound of formula II with a reducing agent and,
b) oxidizing with oxidizing agent to form sulphone of compound III.

In still another embodiment of the present disclosure, the reducing agent is borane complex of dimethyl sulphide.

In still another embodiment of the present disclosure, the oxidizing agent is selected from a group comprising of Sodium hypochlorite/acetic acid, Hydrogen peroxide/acetic acid, Oxone, sodium periodate, meta chloroperbenzoic acid, KMNO4, and similar oxidizing agents.

In still another embodiment of the present disclosure, the compound of formula III is optionally reacted with Boc protected ethylene diamine in a suitable solvent to obtain compound IV.

In still another embodiment of the present disclosure, the compound of formula III is optionally reacted with 2-azido ethylamine in a suitable solvent to obtain compound IV.

In still another embodiment of the present disclosure, the Boc protected compound IV is treated with acetyl chloride in a suitable solvent to obtain Caspofungin.

In still another embodiment of the present disclosure, the azide protected compound IV is hydrogenated to obtain Caspofungin.

In still another embodiment of the present disclosure, hydrogenation is done by using Pd/C in a suitable solvent; the process further comprises a process for preparation of Caspofungin diacetate salt.

In still another embodiment of the present disclosure, Caspofungin is treated with acetic acid in a suitable solvent.

In still another embodiment of the present disclosure, preparation of compound IV further comprises;
(i) reacting pneumocandin Bo with metal halide in presence of an acid in a suitable solvent,
(ii) reacting compound from step (i) with boran complex and
(iii) reacting with NH2CH2CH2-Z.

In still another embodiment of the present disclosure, the metal halide is selected from a group of alkaline or alkaline earth metal halide.

In still another embodiment of the present disclosure, the metal halide is selected from a group comprising of KBr, KI, NaBr, LiBr, LiI, NaI and mixtures thereof.

In still another embodiment of the present disclosure, the acid is lewis acid.

In still another embodiment of the present disclosure, lewis acid is selected from the group comprising of BF3 etherate, Methoxy diethylboronate, triethyl borate, Tin (IV) chloride and mixtures thereof.

In still another embodiment of the present disclosure, boran complex is borane complex of dimethyl sulphide.

In still another embodiment of the present disclosure, Z is NHBOC or N₃.

In still another embodiment of the present disclosure, suitable solvent is selected from a group comprising of acetonitrile, ethylene diamine, methanol, acetic acid, THF, ethanol, isopropanol and mixtures thereof.

The present disclosure is also in relation to a compound of formula II

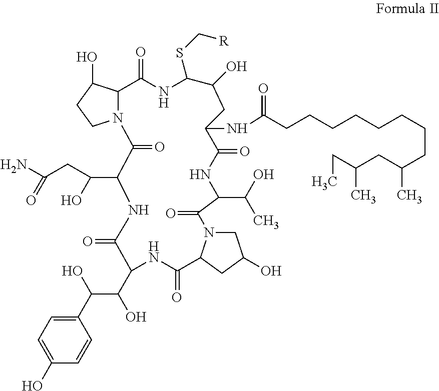

Wherein, R=any side chain consisting of heteroatom.

The present disclosure is also in relation to a compound of formula III

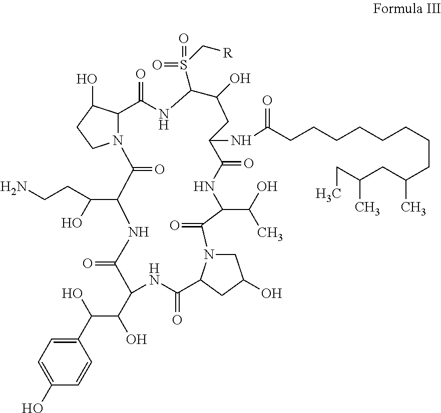

Wherein, R is any side chain consisting of heteroatom.

The present disclosure is also in relation to a compound of formula IV

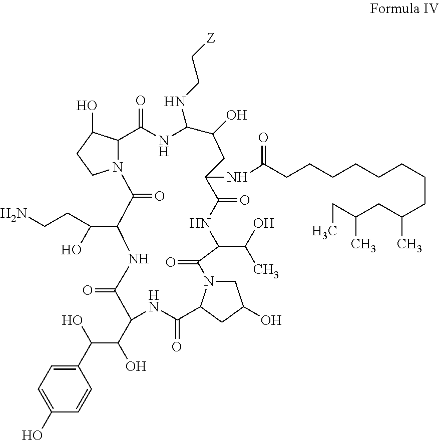

Wherein, Z═NHBOC or N₃.

In an embodiment of the present disclosure, the present disclosure provides a process for preparing Caspofungin acetate by reacting pneumocandin B₀ and derivatives of pneumocandin B₀. The present disclosure provides a novel and more efficient process for the preparation of Caspofungin and novel intermediates thereof. Majority of the intermediates from the present novel process for the preparation of Caspofungin are solids, they are easy to purify and handle. In another embodiment, the present novel process requires less amounts of low cost reagents, which are easy to handle compared to any other existing process. Further, the present novel process for the preparation of Caspofungin and novel intermediates requires less reaction time. In another embodiment, the present disclosure produces less toxic water soluble by-products, which are easy to remove/destroy. The processes resulted from the present disclosure are easy to scale up.

In another embodiment of the disclosure, the pneunocandin B₀ or derivatives are reacted with thiol compounds in anhydrous condition. This reaction is carried out by using the reagents methoxy diethyl borane or triethyl borate or mixtures thereof and triflic acid. The resulted compound is optionally reduced by employing boron dimethyl sulphide complex and further oxidized to get the sulphone of compound III. The reaction further proceeded by reacting the resulted intermediate with N-Boc ethylene diamine and followed by deprotection to form Caspofungin.

In yet another embodiment of the present disclosure, the intermediate which is the sulphone of compound III is treated with 2-azido ethylamine followed by hydrogenation using Pd/C results Caspofungin.

In still another embodiment of the present disclosure, the pneumocandin B₀ is reduced using borane dimethylsulfide complex and treated with metal halide in the presence of a lewis acid to result compound of formula V which is further treated with N-Boc ethylene diamine followed by deprotection yields pure Caspofungin. In another attempt of preparation of caspofungin, compound V is treated with 2-azido ethylamine The resulted intermediate is hydrogenated with Pd/C to yield Caspofungin. Metal halide is selecting from KBr, KI, NaBr, LiBr, LiI, NaI and mixtures thereof. In this process lewis acid is selected from the group of BF3 etherate, Methoxy diethylboronate, triethyl borate, Tin (IV) chloride and mixtures thereof.

In still another embodiment of the present disclosure, the oxidizing agents are selecting from Sodium hypochlorite/acetic acid, Hydrogen peroxide/acetic acid, Oxone, sodium periodate, meta chloroperbenzoic acid, KMNO4, and other similar oxidizing agents.

### EXAMPLES

### Example 1

### Preparation of Ethane Sulfide (Compound II)

To a solution of Pneumocandin B₀ (250 g) in acetonitrile (7.5 L), methoxy diethylborane (110 ml) and ethanethiol (52 ml) were added at room temperature under nitrogen. The reaction mass was stirred for 30 mins. To the reaction mixture Triflic acid (62 ml) was added at −20° C. and stirred for 1 h. After the completion of the reaction, sodium acetate solution was added and stirred for complete precipitation. Filtered the solid, washed with acetonitrile (250 ml) and dried under vacuum to obtain ethane sulfide compound II (180 g).

### Example 2

### Preparation of Compound III from II

a). To a solution of compound II (100 g) in tetrahydrofuran (7 L) borane dimethylsulfide complex (48 ml) was added at −5° C. and stirred for 24 h. After the completion of the reaction methanol (100 ml) was added and concentrated to dryness.
b). To a solution of oxone (64 g) in water (1.8 L), compound obtained from step a (50 g) in methanol (1.8 L) was added at 0° C. and then stirred for completion of the reaction. The resulted solid was filtered, washed with water (1 L) and dried under vacuum to obtain compound III.

### Example 3

### Preparation of Caspofungin Acetate from Compound III

Ethylene diamine (89 ml) in methanol (120 ml) was added to a solution of compound III (30 g) in methanol (120 ml) at 10° C. and stirred for 4 h. After the completion of the reaction, acetic acid (165 ml) was added followed by water (100 ml) and concentrated the reaction mass to obtain caspofungin acetate solution.

### Example 4

### Preparation of Caspofungin Acetate Via Compound V

Potassium bromide (67 g) was added to a solution of Pneumocandin B₀ (20 g) in acetonitrile (60 ml) and boron triflouride diethylether complex (8 g) at −15° C. under nitrogen and stirred for 3 h to obtain compound V. N-Boc ethylene diamine was added to the reaction mixture and then stirred at 30° C. for 12 h. Water (95 ml) was added after the completion of reaction, further reaction mass was concentrated to dryness under vacuum to obtain thick mass. This thick mass was dissolved in tetrahydrofuran (600 ml), borane dimethylsulfide complex (2.5 g) was added and stirred for 24 h at −5° C. Methanol (100 ml) was added after the completion of the reaction, and concentrated to dryness under vacuum. The crude mass was dissolved in methanol (600 ml) and acetyl chloride was added at 0° C. After the completion of the reaction, concentrated the mass and purified to obtain caspofungin acetate (1 g).

### Example 5

### Preparation of Caspofungin Acetate by N—BOC Deprotection

To a solution of Compound IV (N—BOC Capsofungin) (5 g) in methanol (600 ml), acetyl chloride (10 ml) was added at 0° C. and stirred vigorously till the completion of the reaction. The resulting mass was concentrated and purified to obtain caspofungin acetate.

### Example 6

### Preparation of Capsofungin Acetate by Azide Reduction

To the crude Compound IV (Caspofungin azide) (5 g), methanol (500 g) was added and stirred vigorously to make uniform solution. 5% Pd/C (100 mg) was added to the reaction mixture under nitrogen, then evacuated with hydrogen, and then stirred the reaction mixture under hydrogen atmosphere for 12 h till the completion of the reaction. Acetic acid (5 ml) was added and the resulting mass was concentrated and purified to obtain caspofungin acetate (2 g).

### Example 7

### Purification of Capsofungin Acetate

The crude Caspofungin acetate (Purity 20%) was passed through micron filter to remove solid particles and then purified by Novasep-LC110 using mobile phase: ACN/Water/0.01% Acetic acid. Pure fractions were pooled and concentrated to 40-50 mg/ml.

We claim:

1. A process preparation of Caspofungin; said process comprising acts of:
(i) conversion of pneumocandin B₀ to compound of formula II in a suitable solvent, 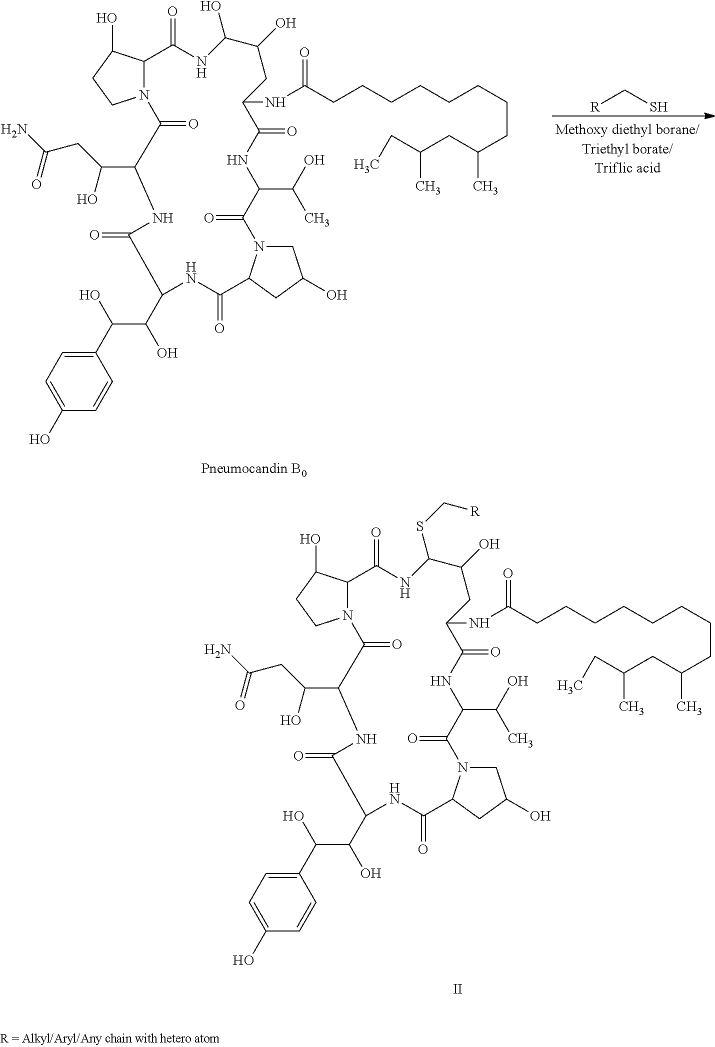
(ii) conversion of formula II to III in a suitable solvent, 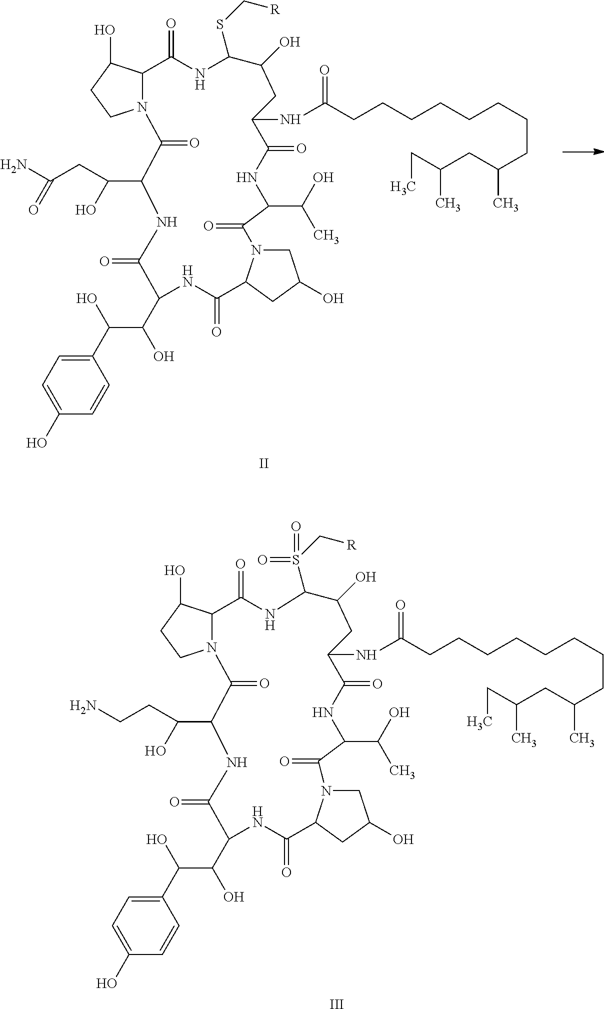
(iii) conversion of formula III to formula IV in a suitable solvent, 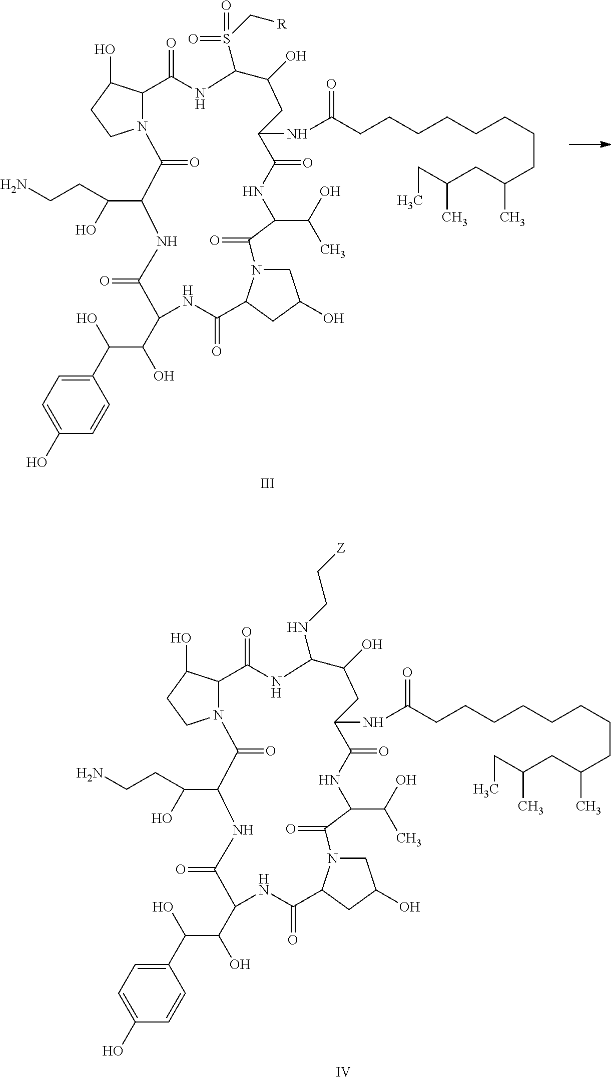
Where Z is NHBOC or N3
(iv) Optionally reacting compound IV with BOC deprotecting reagent in a suitable solvent to isolate Caspofungin salt wherein Z is NHBOC
(v) Optionally hydrogenating compound of formula IV in a suitable solvent to isolating the caspofungin salt wherein Z is N₃.

2. The process as claimed in claim 1, wherein in the step (i) pneumocandin B₀ is reacted with RCH2SH in presence of alkoxy dialkylborane or trialkyl borate or mixture thereof, and triflic acid in a suitable solvent to obtain compound of the formula II.

3. The process as claimed in claim 2, wherein R is selected from a group comprising of alkyl, aryl or any chain comprising a hetero atom, or mixture thereof.

4. The process as claimed in claim 2, wherein alkoxy dialkylborane is methoxy diethylborane.

5. The process as claimed in claim 2, wherein trialkyl borate is triethyl borate.

6. The process as claimed in claim 1, wherein conversion of compound II to compound III further comprises;
a) reducing compound of formula II with a reducing agent and,
b) oxidizing with oxidizing agent to form sulphone of compound III.

7. The process as claimed in claim 6, wherein the reducing agent is borane complex of dimethyl sulphide.

8. The process as claimed in claim 6, wherein the oxidizing agent is selected from a group comprising of Sodium hypochlorite/acetic acid, Hydrogen peroxide/acetic acid, Oxone, sodium periodate, meta chloroperbenzoic acid, KMNO4, and similar oxidizing agents.

9. The process as claimed in claim 1, wherein the compound of formula III is optionally reacted with Boc protected ethylene diamine in a suitable solvent to obtain compound IV.

10. The process as claimed in claim 1, wherein the compound of formula III is optionally reacted with 2-azido ethylamine in a suitable solvent to obtain compound IV.

11. The process as claimed in claim 9, wherein the Boc protected compound IV is treated with acetyl chloride in a suitable solvent to obtain Caspofungin.

12. The process as claimed in claim 10, wherein the azide protected compound IV is hydrogenated to obtain Caspofungin.

13. The process as claimed in claim 12, wherein hydrogenation is carried out by using Pd/C in a suitable solvent.

14. The process as claimed in claim 1 further comprises a for preparation of Caspofungin diacetate salt.

15. The process as claimed in claim 14, wherein Caspofungin is treated with acetic acid in a suitable solvent.

16. The process as claimed in claim 1, wherein preparation of compound IV further comprises of:
(i) reacting pneumocandin Bo with metal halide in presence of an acid in a suitable solvent,
(ii) reacting compound from step (i) with boran complex and
(iii) reacting with NH₂CH₂CH₂—Z to obtain compound IV.

17. The process as claimed in claim 16, wherein the metal halide is selected from a group comprising alkaline or alkaline earth metal halide.

18. The process as claimed in claim 17, wherein the metal halide is selected from a group comprising KBr, KI, NaBr, LiBr, LiI, NaI and mixtures thereof.

19. The process as claimed in claim 16, wherein the acid is lewis acid.

20. The process as claimed in claim 19, wherein lewis acid is selected from the group comprising BF3 etherate, Methoxy diethylboronate, triethyl borate, Tin (IV) chloride and mixtures thereof.

21. The process as claimed in claim 16, wherein boran complex is borane complex of dimethyl sulphide.

22. The process as claimed in claim 16, wherein Z is NHBOC or N₃.

23. The process as claimed in claim 1, wherein the suitable solvent is selected from a group comprising acetonitrile, ethylene diamine, methanol, acetic acid, THF, ethanol, isopropanol and mixtures thereof.

24. A compound of formula II, 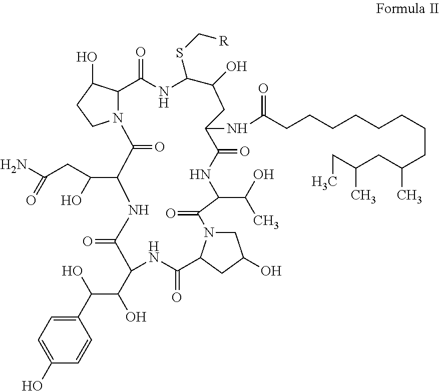
Wherein, R=any side chain consisting of heteroatom.
A compound of formula III, 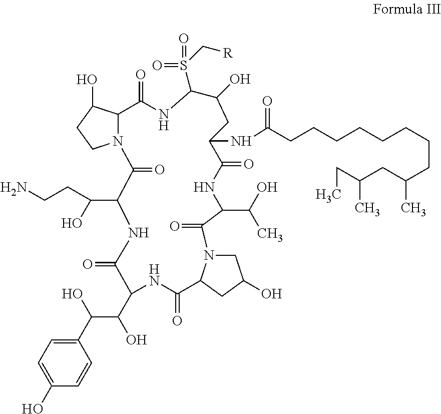
Wherein, R is any side chain consisting of heteroatom.

25. A compound of formula IV, 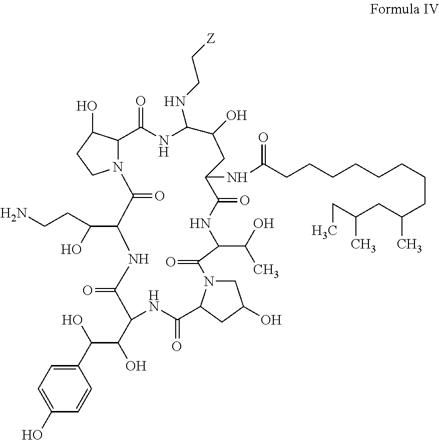
Wherein, Z═NHBOC or N₃.
